(19) United States
(12) Patent Application Publication
(10) Pub. No.: US 2020/0040112 A1
(43) Pub. Date: Feb. 6, 2020

(54) ACRYLIC DERIVATIVES OF 1,4:3,6-DIANHYDROHEXITOL

(71) Applicant: ROQUETTE FRERES, Lestrem (FR)

(72) Inventors: Clothilde BUFFE, Lomme (FR); Jean-Marc CORPART, Lambersart (FR); Vincent WIATZ, Premesques (FR)

(21) Appl. No.: 16/499,053

(22) PCT Filed: Mar. 27, 2018
(86) PCT No.: PCT/FR2018/050750

§ 371 Date: Sep. 27, 2019

(30) Foreign Application Priority Data
Mar. 28, 2017 (FR) 17 52560

(51) Int. Cl. C08F 20/28; C07D 493/04

(52) U.S. Cl. CPC C08F 20/28; C07D 493/04

(57) ABSTRACT

The invention relates to a compound of formula I

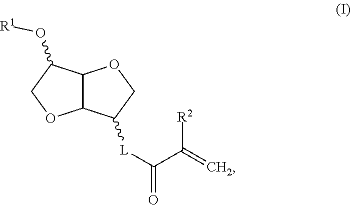

the preparation thereof and the use thereof as monomer for the preparation of polymers.

### FIELD OF THE INVENTION

The invention relates to novel acrylic derivatives of 1,4:3,6-dianhydrohexitol which are especially useful for producing polymers.

### PRIOR ART

Numerous industries require compositions making it possible to produce polymers, for example in the form of bulk materials or coatings. In the latter case, these may for example be protective, decorative or surface-treatment coatings. A large number of crosslinkable compositions already exist for this purpose in the literature and commercially. These compositions mostly consist of a mixture of polymerizable monomers produced by the chemical industry from petroleum derivatives.

However, in the current context of the gradual decrease in petroleum product resources, it is increasingly advantageous to replace products of petroleum origin with products of natural origin.

The use of biobased polymers, i.e. polymers produced from raw materials of natural origin, has already been described. In particular, the publication by L. Jasinska and C. E. Koning (“Unsaturated, biobased polyesters and their cross-linking via radical copolymerization”, Journal of Polymer Science Part A: Polymer Chemistry, Volume 48, Issue 13, pages 2885-2895, 1 Jul. 2010) describes the preparation of unsaturated polyesters having relatively high glass transition temperatures (T_{g}), i.e. greater than 45° C., these polyesters being particularly useful for the production of coatings. These unsaturated polyesters are obtained by polymerization of isosorbide with maleic anhydride and optionally succinic acid. In order to be able to be used as a coating, these unsaturated polyesters must, however, be crosslinked. The polymers may either be dissolved or suspended with a crosslinking agent, then applied to the substrate, and finally crosslinked after evaporation of the solvent, or deposited by standard “powder coating” techniques, or melted to be deposited. In all cases, these deposition techniques require a post-crosslinking step. The fact that the production of the coating requires several successive steps is restrictive.

In order to overcome these disadvantages, the use of di(meth)acrylic derivatives of isosorbide as crosslinkable monomers has been proposed. Acrylate and methacrylate derivatives of isosorbide were described for the first time by Wiggins et al. in 1946 (GB 586141). The patent application WO 2014/147340 A1 in the name of the Applicant describes crosslinkable compositions comprising isosorbide diacrylate or isosorbide dimethacrylate. Patent application WO 2015/004381 A1, also in the name of the Applicant, describes an isosorbide caprolactate diacrylate which would be useful in the production of polymers. However, these isosorbide dimethacrylate and diacrylates have the disadvantage that the resins obtained therewith have a high degree of crosslinking, which results in brittle coatings.

Isosorbide mono(meth)acrylate derivatives, the second hydroxyl function of which is substituted or unsubstituted, are also known. Patent application US 2016/0139526 A1 for example describes resins based on isosorbide (meth)acrylate and the use thereof in toner compositions, this (methacrylate) possibly being monosubstituted.

Patent application US 2013/0017484 relates to highly specific monoacrylate derivatives of isosorbide, the second hydroxyl group of which is substituted with an acid-labile group or an acetal function. These compounds are useful for the preparation of polymers transparent to radiation≤500 nm.

Patent application US 2016/0229863 A1 and the article by Gallagher et al. (ACS Sustainable Chem Eng., 2015, 3, 662-667) describe the synthesis of isosorbide monomethacrylate monoacetate derivatives. They are prepared via reaction between isosorbide monoacetate and methacrylic anhydride in the presence of scandium triflate. The products, purified by column chromatography, are viscous liquids. No significant difference was observed between the two exo and endo monoacetate isomers. Polymerization of these monomers resulted in materials having a high glass transition temperature (Tg) (130° C.) and thermal stability similar to PMMA.

One of the aims of the present invention is to propose novel mono(meth)acrylate derivatives of 1,4:3,6-dianhydrohexitol.

### SUMMARY OF THE INVENTION

Thus, a subject of the invention is a compound of formula I:

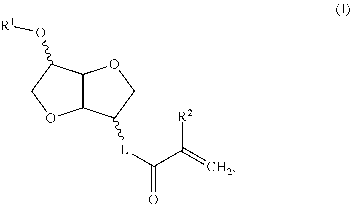

in which
R¹ is a linear or branched C1 to C6 alkyl group,
R² is H or C1 or C2 alkyl,
L is O, —O—CH₂—CH(OH)—CH₂—O—, —O—C(O)—NH-L¹-O— in which L¹ is chosen from linear or branched alkylene, or —O—C(O)—NH-L²-O-L³-O— in which —O—C(O)—NH-L²- is a residue of a reagent chosen from isophorone diisocyanate (IPDI), IPDI isocyanurate, polymeric IPDI, 1,5-naphthalene diisocyanate (NDI), methylenebis-cyclohexylisocyanate, methylene diphenyl diisocyanate (MDI), polymeric MDI, toluene diisocyanate (TDI), TDI isocyanurate, the TDI-trimethylolpropane adduct, polymeric TDI, hexamethylene diisocyanate (HDI), HDI isocyanurate, HDI biurate, polymeric HDI, xylylene diisocyanate, hydrogenated xylylene diisocyanate, tetramethyl xylylene diisocyanate, /7-phenylene diisocyanate, 3,3′-dimethyldiphenyl-4,4′-diisocyanate (DDDI), 2,2,4-trimethylhexamethylene diisocyanate (TMDI), norbornane diisocyanate (NDI) and 4,4′-dibenzyl diisocyanate (DBDI), and L3 is chosen from linear or branched alkylene and poly(propylene glycol).

### DETAILED DESCRIPTION OF THE INVENTION

Preferred compounds of formula (I) are those in which one or more or even each of R¹, R² and L is defined as follows:

R¹ is a linear or branched C1 to C4 alkyl group, preferably R¹ is methyl or ethyl, more preferably still R¹ is methyl;
R² is H or methyl;
L is O, —O—CH₂—CH(OH)—CH₂—O—, —O—C(O)—NH-L¹-O— in which L¹ is chosen from linear or branched C2 to C4 alkylene, or —O—C(O)—NH-L²-O-L³-O— in which —O—C(O)—NH-L²- is a residue of a reagent chosen from isophorone diisocyanate (IPDI), IPDI isocyanurate, polymeric IPDI, 1,5-naphthalene diisocyanate (NDI), methylenebis-cyclohexylisocyanate, methylene diphenyl diisocyanate (MDI), polymeric MDI, toluene diisocyanate (TDI), TDI isocyanurate, the TDI-trimethylolpropane adduct, polymeric TDI, hexamethylene diisocyanate (HDI), HDI isocyanurate, HDI biurate, polymeric HDI, xylylene diisocyanate, hydrogenated xylylene diisocyanate, tetramethyl xylylene diisocyanate, /7-phenylene diisocyanate, 3,3′-dimethyldiphenyl-4,4′-diisocyanate (DDDI), 2,2,4-trimethylhexamethylene diisocyanate (TMDI), norbornane diisocyanate (NDI) and 4,4′-dibenzyl diisocyanate (DBDI), and L3 is chosen from linear or branched, preferably C2 to C4, alkylene and poly(propylene glycol), L is preferably O, —O—CH₂—CH(OH)—CH₂—O— or —C(O)—NH-L¹-O— in which L¹ is linear or branched C2 to C4 alkylene, more preferably still L is O, —O—CH₂—CH(OH)—CH₂—O— or —O—C(O)—NH—(CH₂)₂—O—, and more preferentially still, L is O or —O—CH₂—CH(OH)—CH₂—O—.

The compounds of formula I described above exist in various conformations due to the presence of the 1,4:3,6-dianhydrohexitol ring system. Thus, the compounds of formula I may be derivatives of isosorbide (1,4:3,6-dianhydro-D-glucitol), isoidide (1,4:3,6-dianhydro-L-iditol) or isomannide (1,4:3,6-dianhydro-D-mannitol). The present invention thus covers the compounds of formulae Ia, Ib, Ic and Id:

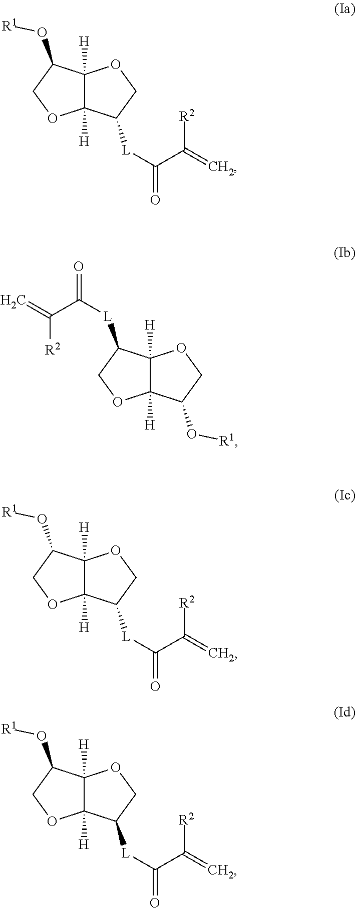

in which R¹, R² and L are as defined above in relation to formula I, and also the mixtures thereof. Preferably, the compound of formula I is chosen from compounds according to formula Ia, Ib and the mixtures thereof.

In one embodiment, the compound of formula I corresponds to formula II:

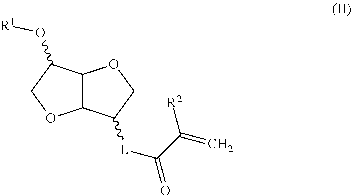

in which R¹ and R² are as defined above in relation to formula I.

Preferably, R² is methyl.

The compound of formula II may be chosen from the compounds of formula IIa, IIb, IIc, and IId:

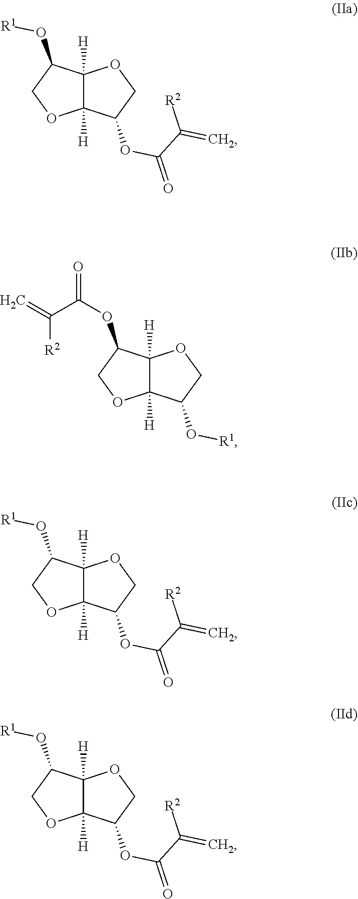

in which R¹ and R² are as defined above in relation to formula II, and also from the mixtures thereof. Preferably, the compound of formula II is chosen from compounds according to formula IIa, IIb and the mixtures thereof.

In another embodiment, the compound of formula I corresponds to formula III:

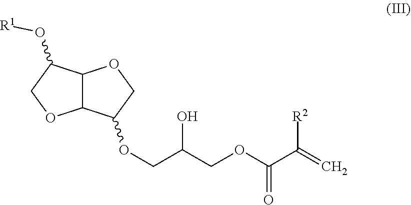

in which R¹ and R² are as defined above in relation to formula I.

Preferably, R² is methyl.

The compound of formula III may be chosen from compounds of formula IIIa, IIIb and IIIc and IIId:

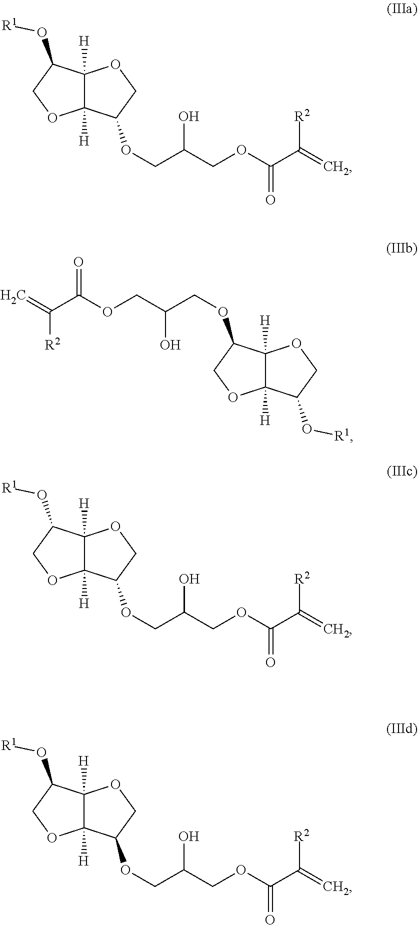

in which R¹ and R² are as defined above in relation to formula III. Preferably, the compound of formula II is chosen from compounds according to formula IIIa, IIIb or the mixtures thereof.

According to one embodiment, the compound according to the invention is a compound according to one of the formulae defined above in which, when L is O, R¹ cannot be a tertiary C4 to C6 alkyl group, especially tert-butyl, and/or when L is O and R² is H, R¹ cannot be methyl.

The compounds of the invention may be prepared according to synthesis methods known to those skilled in the art. They may for example be prepared by a two-step synthesis process from 1,4:3,6-dianhydrohexitol comprising a first step of protecting a hydroxyl group of the 1,4:3,6-dianhydrohexitol with an ether function and a second step of functionalizing the other hydroxyl group with an acrylate function.

More particularly, the compounds of formula I may be prepared by a process comprising the following steps:
a) preparation of a linear or branched C1 to C6 aliphatic monoether of 1,4:3,6-dianhydrohexitol by reacting the 1,4:3,6-dianhydrohexitol with an alkylating agent;
b) functionalization of the free hydroxyl group of the monoether obtained in step a) with an acrylate, methacrylate, epoxy acrylate, epoxy methacrylate, isocyanate acrylate or isocyanate methacrylate function.

The 1,4:3,6-dianhydrohexitol may be chosen from isosorbide (1,4:3,6-dianhydro-D-glucitol), isoidide (1,4:3,6-dianhydro-L-iditol) and isomannide (1,4:3,6-dianhydro-D-mannitol). The preferred 1,4:3,6-dianhydrohexitol is isosorbide. When the 1,4:3,6-dianhydrohexitol is isosorbide, at the end of step b) a compound of formula Ia, Ib or a mixture of the two is obtained. In the case in which a mixture of compounds of formulae Ia and Ib is obtained, this mixture may be separated by techniques known to those skilled in the art, for instance by column chromatography.

The preparation of the monoether in step a) may be carried out according to methods known to those skilled in the art, for example from 1,4:3,6-dianhydrohexitol and a linear or branched C1 to C6 alkylating agent. The linear or branched C1 to C6 alkyl residue of the alkylating agent is advantageously chosen from linear or branched C1 to C4 alkyl residues, preferably from methyl and ethyl. More preferably still, the alkyl residue is methyl. Etherification reactions that may be used are for example described in patent applications WO2014023902 A1, WO 2014/168698 A1 and WO 2016/156505 A1.

The alkylating agent may especially be chosen from linear or branched C1 to C6 aliphatic alcohols, linear or branched C1 to C6 aliphatic alkyl halides, linear or branched C1 to C6 aliphatic esters of sulfuric acids, linear or branched C1 to C6 aliphatic dialkyl carbonates or linear or branched C1 to C6 aliphatic dialkoxymethanes. As specified above, the C1 to C6 alkyl residues are advantageously chosen from linear or branched C1 to C4 alkyl residues, preferably from methyl and ethyl. More preferably still, the alkyl residue is methyl.

The alcohols that may be used as alkylating agent comprise in particular methanol, ethanol, isopropanol and tert-butanol, methanol being preferred. The alkyl halides that may be used as alkylating agent comprise in particular methyl, ethyl, isopropyl and tert-butyl halides, methyl halides being preferred. The linear or branched C1 to C6 aliphatic esters of sulfuric acids may for example be chosen from methyl, ethyl, isopropyl and tert-butyl esters, methyl esters, in particular dimethyl sulfate, being preferred. The dialkyl carbonates may for example be chosen from dimethyl carbonate, diethyl carbonate, diisopropyl carbonate and di-tert-butyl carbonate, dimethyl carbonate being preferred. The dialkoxymethanes that may be used as alkylating agent comprise in particular dimethoxymethane, diethoxymethane, diisopropoxymethane and di-tert-butoxymethane, dimethoxymethane being preferred.

The 1,4:3,6-dianhydrohexitol may be chosen from isosorbide (1,4:3,6-dianhydro-D-glucitol), isoidide (1,4:3,6-dianhydro-L-iditol) and isomannide (1,4:3,6-dianhydro-D-mannitol). The preferred 1,4:3,6-dianhydrohexitol is isosorbide. When the 1,4:3,6-dianhydrohexitol is isosorbide, a compound of formula Ia, Ib (or sub-formulae IIa and IIb or IIIa and IIIb), or a mixture of both, is obtained at the end of step b). In the case in which a mixture of compounds of formulae Ia and Ib (or sub-formulae IIa and IIb or IIIa and IIIb) is obtained, this mixture may be separated by techniques known to those skilled in the art, for instance by column chromatography.

At the end of step a), a mixture of monoalkyl ether, dialkyl ether and dianhydrohexitol is generally obtained which can be used directly in step b) or the mixture can be separated by distillation with rectification under reduced pressure before step b).

In step b), the free hydroxyl group is functionalized with an acrylate, methacrylate, epoxy acrylate, epoxy methacrylate, isocyanate acrylate or isocyanate methacrylate function, preferably with an acrylate, methacrylate, epoxy acrylate or epoxy methacrylate function.

The functionalization of the free hydroxyl group with an acrylate, methacrylate, epoxy acrylate, epoxy methacrylate, isocyanate acrylate or isocyanate methacrylate function may be carried out according to methods known to those skilled in the art. Use may for example be made of acrylic and methacrylic acids, acrylic and methacrylic acid esters, acrylic and methacrylic anhydrides, glycidyl acrylate, glycidyl methacrylate, alkylisocyanate acrylates and methacrylates, or diisocyanates with hydroxyalkyl acrylates or hydroxyalkyl methacrylates.

When the free hydroxyl group is functionalized with an acrylate or methacrylate function, compounds of formula II are obtained. In this case, the free hydroxyl group may be reacted with acrylic or methacrylic acid, an acrylic or methacrylic acid ester, or an acrylic or methacrylic anhydride.

When the free hydroxyl group is functionalized with an epoxy acrylate or epoxy methacrylate function, compounds of formula III are obtained. In this case, the free hydroxyl group may be reacted with glycidyl acrylate and glycidyl methacrylate.

The hydroxyl group may also be functionalized with an isocyanate acrylate or isocyanate methacrylate function. In this case, the free hydroxyl group may be reacted with an alkylisocyanate acrylate or methacrylate, in particular with a linear or branched C2 to C4 alkylisocyanate acrylate or methacrylate, preferably with an ethylisocyanate acrylate or methacrylate. It is also possible to proceed in two steps, by firstly reacting the free hydroxyl group with a diisocyanate then by reacting the product thus obtained with a hydroxyalkyl acrylate, a hydroxyalkyl methacrylate, a poly(propylene glycol) acrylate or a poly(propylene glycol) methacrylate. Regarding the hydroxyalkyl acrylates and methacrylates, use will advantageously be made of those which are linear or branched, C2 to C4, preferably hydroxyethyl acrylate or methacrylate. Diisocyanate, as used here, is intended to mean a compound comprising at least two isocyanate functions. This diisocyanate may for example be chosen from isophorone diisocyanate (IPDI), IPDI isocyanurate, polymeric IPDI, 1,5-naphthalene diisocyanate (NDI), methylenebis-cyclohexylisocyanate, methylene diphenyl diisocyanate (MDI), polymeric MDI, toluene diisocyanate (TDI), TDI isocyanurate, the TDI-trimethylolpropane adduct, polymeric TDI, hexamethylene diisocyanate (HDI), HDI isocyanurate, HDI biurate, polymeric HDI, xylylene diisocyanate, hydrogenated xylylene diisocyanate, tetramethyl xylylene diisocyanate, /7-phenylene diisocyanate, 3,3′-dimethyldiphenyl-4,4′-diisocyanate (DDDI), 2,2,4-trimethylhexamethylene diisocyanate (TMDI), norbornane diisocyanate (NDI) and 4,4′-dibenzyl diisocyanate (DBDI).

The compounds of the invention may be used for the preparation of thermoplastic acrylic resins. In particular, they may partially or completely replace the methyl methacrylate in polymers of the PMMA (polymethyl methacrylate) type, better known under the name PLEXIGLAS®.

They may be used alone or in combination with numerous other monomers capable of integrating into a radical polymerization process, for example acrylic and methacrylic monomers (methyl methacrylate, butyl acrylate, glycidyl methacrylate, etc.), styrene and vinyl acetate.

Prepared in bulk, in solution or in dispersion (emulsion, suspension, etc.) these resins can then be used to produce films or materials that can be used in the field of coatings (paint, ink, etc.), adhesives, dental prostheses, optical materials, excipients for pharmaceuticals, etc.

These compounds can also be used as reactive diluent and/or flexibilizing agent in the preparation of thermosetting resins optionally in combination with other monomers and in particular monofunctional and/or multifunctional acrylates and/or styrene.

The invention will now be illustrated in the examples hereinafter. It is specified that these examples do not in any way limit the present invention.

### EXAMPLES

### Example 1: Synthesis of Isosorbide Methyl Ether

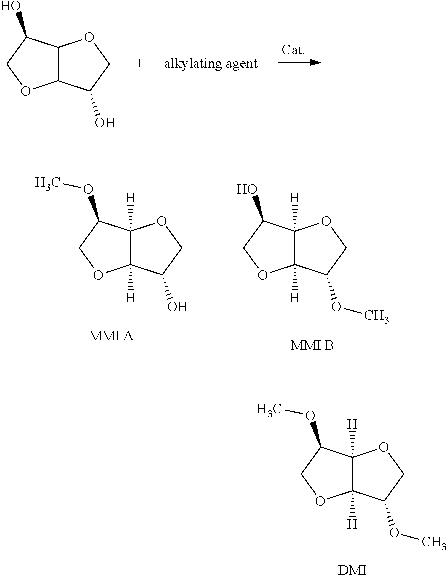

500 g of isosorbide and 125 g of water are charged in a 2 l jacketed reactor surmounted by a condenser, fitted with a mechanical stirrer and a thermometer. The medium is heated to 50° C. then 258.9 g of dimethyl sulfate and 172.4 g of 50% sodium hydroxide are introduced simultaneously using peristaltic pumps and taking care not to exceed 65° C. in the medium. The addition lasts 2 hours.

As soon as the addition is complete, the medium is brought to 95° C. then 172.4 g of sodium hydroxide are introduced in 2 hours using a peristaltic pump.

The reaction medium is subsequently kept stirring at 95° C. for at least 3 h.

After filtration and concentration on a rotary evaporator, the product is obtained in liquid form, contains 19.7% of isosorbide, 20.5% of isosorbide 5-O-monomethyl ether A (MMI A-functionalization in the endo position), 24.8% of isosorbide 2-O-monomethyl ether (MMI B-functionalization in the exo position) and 25% isosorbide dimethyl ether (DMI). The percentages correspond to percentages by weight measured by NMR analysis.

### Example 2: Synthesis of Isosorbide Ethyl Ether

500 g of isosorbide and 125 g of water are charged in a 2 l jacketed reactor surmounted by a condenser, fitted with a mechanical stirrer and a thermometer. The medium is heated to 50° C. then 316.1 g of diethyl sulfate and 172.4 g of 50% sodium hydroxide are introduced simultaneously using peristaltic pumps and taking care not to exceed 65° C. in the medium. The addition lasts 2 hours.

As soon as the addition is complete, the medium is brought to 95° C. then 172.4 g of sodium hydroxide are introduced in 2 hours using a peristaltic pump.

The reaction medium is subsequently kept stirring at 95° C. for at least 3 h.

After filtration and concentration on a rotary evaporator, the product is obtained in liquid form, contains 18% of isosorbide, 21.4% of isosorbide 5-O-monoethyl ether (MEI A), 26.2% of isosorbide 2-O-monoethyl ether (MEI B) and 25.6% isosorbide diethyl ether (DEI). The percentages correspond to percentages by weight measured by NMR analysis.

### Example 3: Obtaining Monomethyl Isosorbide

1641.9 g of the product obtained according to example 1 are introduced into a 2 l jacketed reactor surmounted by a rectifying column, a reflux head, a condenser and recovery receivers. The rectifying column is filled with 10 Sulzer EX type packing elements.

First, the assembly is placed under reduced pressure (15 mbar) and the product is heated at 150° C. under total reflux until the temperatures at the top of the column stabilize.

As soon as the temperatures are stabilized, a first distillation fraction is obtained with an imposed reflux ratio of greater than 1.

While carrying out the rectification, the pressure is gradually reduced down to 0.4 mbar and the temperature of the medium is increased up to 200° C.

The distillation temperatures at the top of the column and the distillation pressures of each compound are collated in the table:

| | | T column | | | NMR purity |
|---|---|---|---|---|---|
| Product | Compound | top (° C.) | P (mbar) | weight (g) | (%) |
| 1 | DMI | 124-129 | 14-16 | 271 | 83.3 |
| 2 | MMI B | 115-121 | 5 | 228 | 93.2 |
| 3 | MMI A | 121-124 | 0.9-1.1 | 238 | 93.3 |
| 4 | Isosorbide | 130 | 0.4 | 23 | 85.3 |

### Example 4: Synthesis of Isosorbide Monomethyl Methacrylate A

40 g of MMI A (product 3), 23.7 g of methacrylic acid and 150 g of xylenes are introduced into a 250 ml three-necked round-bottomed flask surmounted by a Dean-Stark apparatus, heated using a heating mantle and equipped with a magnetic stirrer.

64 mg of phenothiazine, 1040 mg of 70% methanesulfonic acid and 110 mg of 50% hypophosphorous acid are added.

The reaction medium is then heated to the boiling point of the solvent for at least 24 h. The water is continuously eliminated by azeotropic distillation.

After 24 hours of reaction, the acid number is 19 mg KOH/g of crude. The product is purified by liquid-liquid extraction. A first wash with a 6% sodium hydroxide solution is carried out, then 2 washes with water.

The organic phase is dried using anhydrous magnesium sulfate, filtered and concentrated using a rotary evaporator after adding 10 mg of hydroquinone monomethyl ether.

35 g of monomethyl methacrylate A are obtained in liquid form. The structure is confirmed by NMR analysis with a purity by weight of greater than 85%.

### Example 5: Synthesis of Isosorbide Monomethyl Methacrylate B

Following the protocol identical to that of example 4, replacing MMI A with MMI B (product 2). 36 g of monomethyl methacrylate B are obtained in solid form. The structure is confirmed by NMR analysis with a purity by weight of greater than 85%.

### Example 6: Synthesis of Isosorbide Monomethyl Methacrylate (Mixture A and B)

11 g of MMI A (product 3), 11 g of MMI B (product 2) and 100 ml of dichloromethane are introduced into a 500 ml jacketed reactor surmounted by a condenser and provided with a magnetic stirrer. The medium is cooled to 0° C.

A solution of trimethylamine (16.7 g in 50 ml of dichloromethane) is then added to the reaction medium. Then a solution of methacryloyl chloride (17.3 g) in dichloromethane (100 ml) is added dropwise using a peristaltic pump taking care not to exceed 5° C. in the medium.

The reaction medium is then kept stirring at room temperature for at least 6 hours.

At the end of the reaction, the reaction medium is filtered then purified by successive washings with saturated aqueous NaHCO₃ solution, NaOH (1M) and NaCl.

The organic phase is dried using anhydrous magnesium sulfate, filtered and concentrated on a rotary evaporator after adding 10 mg of hydroquinone monomethyl ether.

The product obtained is a slightly colored liquid. The structure is confirmed by NMR analysis with a purity by weight of greater than 85%.

### Example 7: Synthesis of Isosorbide Monomethyl Urethane-Methacrylate B

36.4 g of isophorone diisocyanate, 25 g of MMIB and 0.1 g of dibutyltin dilaurate are introduced into a 250 ml jacketed reactor.

The reaction medium is heated to 75° C. and kept stirring for at least 4 h.

21.4 g of 2-hydroxyethyl methacrylate are then introduced and the medium is kept stirring for at least 3 h at 60° C.

The disappearance of the NCO group is monitored by Infrared analysis (peak at 2200 cm⁻¹).

### Example 8: Synthesis of Isosorbide Monomethyl Epoxy-Methacrylate B

20 g of isosorbide monomethyl ether MMIB (0.125 mol, 1 eq) and 12.65 g of triethylamine (0.125 mol, 1 eq) in 180 g of dichloromethane are introduced into a 250 ml jacketed reactor fitted with a mechanical stirrer and a condenser. The assembly is placed under a light stream of nitrogen.

17.8 g of glycidyl methacrylate (0.125 mol, 1 eq) are then introduced dropwise. As soon as the addition is complete, the medium is heated to 70° C. by means of a thermostatic bath.

The reaction is monitored by NMR analysis. At the end of the reaction, the medium is purified by liquid/liquid water/dichloromethane extraction. The organic phase is then dried with magnesium sulfate anhydride and concentrated on a rotavap.

The crude product is then purified by silica column chromatography (eluent ethyl acetate/cyclohexane).

The product obtained is a slightly colored liquid. The structure is confirmed by NMR analysis.

1. A compound of formula I 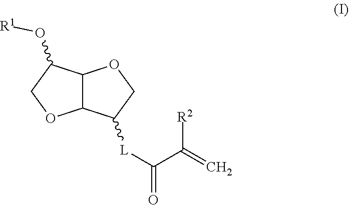
in which
R¹ is a linear or branched C1 to C6 alkyl group,
R² is H or C1 or C2 alkyl,
L is O, —O—CH₂—CH(OH)—CH₂—O—, —O—C(O)—NH-L¹-O— in which L¹ is chosen from linear or branched alkylene, or —O—C(O)—NH-L²-O-L³-O— in which —O—C(O)—NH-L²- is a residue of a reagent chosen from isophorone diisocyanate (IPDI), IPDI isocyanurate, polymeric IPDI, 1,5-naphthalene diisocyanate (NDI), methylenebis-cyclohexylisocyanate, methylene diphenyl diisocyanate (MDI), polymeric MDI, toluene diisocyanate (TDI), TDI isocyanurate, the TDI-trimethylolpropane adduct, polymeric TDI, hexamethylene diisocyanate (HDI), HDI isocyanurate, HDI biurate, polymeric HDI, xylylene diisocyanate, hydrogenated xylylene diisocyanate, tetramethyl xylylene diisocyanate, /7-phenylene diisocyanate, 3,3′-dimethyldiphenyl-4,4′-diisocyanate (DDDI), 2,2,4-trimethylhexamethylene diisocyanate (TMDI), norbornane diisocyanate (NDI) and 4,4′-dibenzyl diisocyanate (DBDI), and L3 is chosen from linear or branched C2 to C4 alkylene and poly(propylene glycol).

2. The compound as claimed in claim 1, wherein
R¹ is a linear or branched C1 to C6 alkyl group,
R² is H or C1 or C2 alkyl,
L is O, —O—CH₂—CH(OH)—CH₂—O— or —O—C(O)—NH—(CH₂)₂—O—.

3. The compound as claimed in claim 1, wherein R¹ is methyl.

4. The compound as claimed in claim 1, wherein R² is H or methyl.

5. The compound as claimed in claim 1, wherein L is O or —O—CH₂—CH(OH)—CH₂—O—.

6. The compound as claimed in claim 1, wherein the compound is chosen from the compounds of formulae Ia, Ib, Ic, Id: 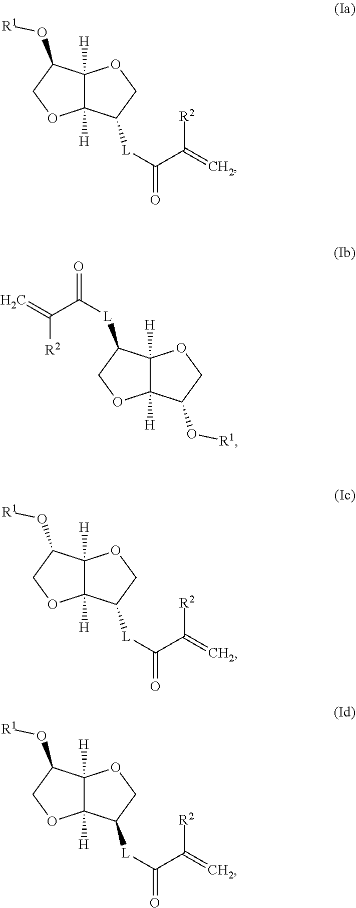
and mixtures thereof.

7. The compound as claimed in claim 1, wherein the compound has the formula II

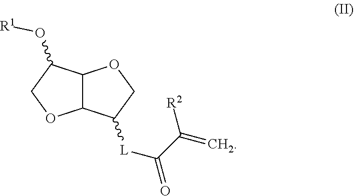

8. The compound as claimed in claim 1, wherein the compound is chosen from the compounds of formulae IIa, IIb, IIc, IId: 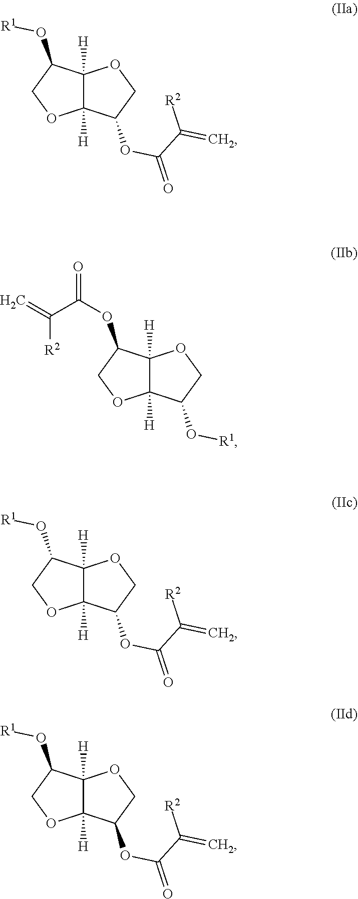
and mixtures thereof.

9. The compound as claimed in claim 1, wherein the compound is chosen from the compounds of formulae IIa, IIb and mixtures thereof.

10. The compound as claimed in claim 1, wherein the compound has the formula III

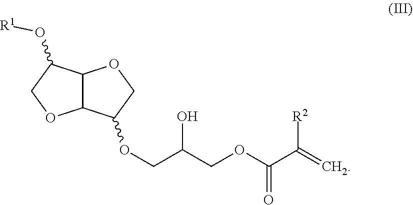

11. The compound as claimed in claim 1, wherein the compound is chosen from the compounds of formulae IIIa, IIIb, IIIc, IIId: 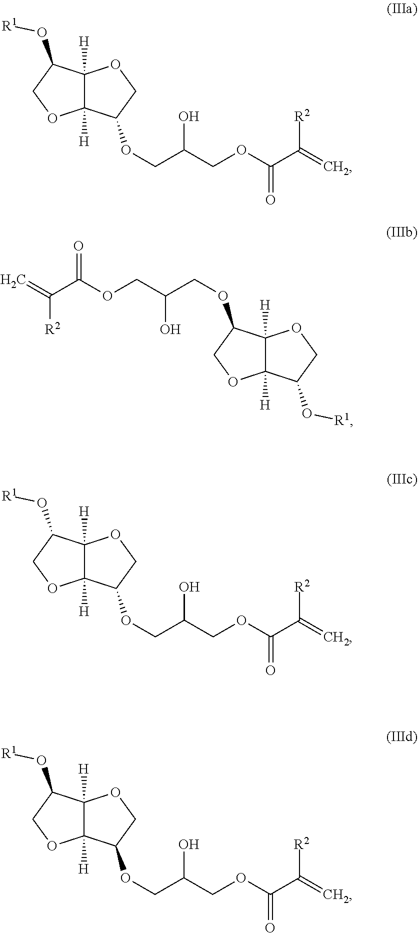
and mixtures thereof.

12. The compound as claimed in claim 1, wherein the compound is chosen from the compounds of formulae IIIa, IIIb and mixtures thereof.

13. A process for producing a compound of formula I as defined in claim 1, comprising the following steps:
a) a linear or branched C1 to C6 alkyl monoether of 1,4:3,6-dianhydrohexitol is prepared by reacting a 1,4:3,6-dianhydrohexitol with an alkylating agent;
b) the free hydroxyl group of the monoether obtained in step a) is functionalized with an acrylate, methacrylate, epoxy acrylate, epoxy methacrylate, isocyanate acrylate or isocyanate methacrylate function.

14. A method of preparation of polymers, comprising a step of polymerization comprising a compound as claimed in claim 1 as monomer.

15. A method of preparation of polymers, comprising a step of polymerization comprising a compound as obtained according to the process of claim 13 as monomer.
